(19) United States
(12) Patent Application Publication
(10) Pub. No.: US 2014/0066934 A1
(43) Pub. Date: Mar. 6, 2014

(54) Bi-Cruciate Knee System

(71) Applicant: Biomet Manufacturing, LLC, Warsaw, IN (US)

(72) Inventors: Carl Deirmengian, Newtown Square, PA (US); Robert Metzger, Wakarusa, IN (US)

(73) Assignee: Biomet Manufacturing, LLC, Warsaw, IN (US)

(21) Appl. No.: 14/013,859

(22) Filed: Aug. 29, 2013

Related U.S. Application Data
(63) Continuation-in-part of application No. 13/470,630, filed on May 14, 2012.
(60) Provisional application No. 61/486,023, filed on May 13, 2011.
(60) Provisional application No. 61/593,521, filed on Feb. 1, 2012.

(51) Int. Cl. A61B 17/17; A61B 17/16

(52) U.S. Cl. CPC A61B 17/1764; A61B 17/1675
USPC 606/83; 606/88

(57) ABSTRACT

A tibial tray configured for use in a bi-cruciate retaining knee procedure can include a medial portion, a lateral portion, and an anterior connecting portion. The medial portion can have a medial superior bearing opposing surface, a medial inferior bone opposing surface, and a medial connecting surface. The lateral portion can have a lateral superior bearing opposing surface, a lateral inferior bone opposing surface, and a lateral connecting surface. The anterior connecting portion can connect the medial and lateral portions and cooperate with the medial and lateral portions to define a slot therebetween. The medial and lateral connecting surfaces can be formed on converging planes from respective inferior surfaces to superior surfaces of the tibial tray.

### CROSS-REFERENCE TO RELATED APPLICATIONS

This application is a continuation-in-part of U.S. patent application Ser. No. 13/470,630 filed on May 14, 2012, which claims the benefit of U.S. Provisional Application Nos. 61/486,023, filed on May 13, 2011 and 61/593,521, filed on Feb. 1, 2012. This application is also a non-provisional of U.S. Patent Application No. 61/776,598 filed on Mar. 11, 2013. The entire disclosures of each of the above applications are incorporated herein by reference.

### FIELD

The following disclosure recites generally to knee surgery and more specifically to instrumentation, implants, and related method for preparing a knee for a bi-cruciate knee implant.

### SUMMARY

This section provides a general summary of the disclosure, and is not a comprehensive disclosure of its full scope or all of its features.

A tibial tray configured for use in a bi-cruciate retaining knee procedure can include a medial portion, a lateral portion, and an anterior connecting portion. The medial portion can have a superior medial bearing opposing surface, an inferior medial bone opposing surface, and a medial connecting surface. The lateral portion can have a superior lateral bearing opposing surface, an inferior lateral bone opposing surface, and a lateral connecting surface. The anterior connecting portion can connect the medial and lateral portions and cooperate with the medial and lateral portions to define a slot therebetween. The medial and lateral connecting surfaces can be formed on converging planes that extend from respective inferior bone opposing surfaces to superior bearing opposing surfaces of the tibial tray.

According to additional features, the medial connecting surface can be chamfered relative to the superior medial bearing opposing surface. The lateral connecting surface can be chamfered relative to the superior lateral bearing opposing surface. The medial connecting surface can define an angle of between 30 degrees and 60 degrees relative to the superior medial bearing opposing surface. The medial connecting surface can define an angle of about 45 degrees relative to the superior medial bearing opposing surface. The lateral connecting surface can define an angle of between 30 degrees and 85 degrees relative to the superior lateral bearing opposing surface. The lateral connecting surface can define an angle of about 45 degrees relative to the superior lateral bearing opposing surface.

According to additional features, the anterior connecting portion can have an inferior anterior bone opposing surface. The anterior connecting portion can further include an anterior connecting surface that extends at an angle between 120 degrees and 150 degrees relative to the inferior anterior bone opposing surface. The medial and lateral connecting surfaces can be configured to overhang respective medial and lateral chamfer walls formed on an anterior cruciate island of a tibia in an implanted position.

A method for preparing a proximal tibia for receipt of a bi-cruciate implant is disclosed. The method can include determining a resection level of the proximal tibia. The resection level can correspond to a medial and lateral tibial plateau. A medial cut can be prepared into the proximal tibia. The medial cut can have an obtuse angle relative to the medial tibial plateau. The medial cut can create a medial chamfer wall on the proximal tibia. A lateral cut can be prepared into the proximal tibia. The lateral cut can have an obtuse angle relative to the lateral tibial plateau. The lateral cut can create a lateral chamfer wall on the proximal tibia.

The method can further include positioning a tibial tray onto the prepared proximal tibia. A medial connecting surface of the tibial tray can overhang the medial chamfer wall of the proximal tibia. A lateral connecting surface of the tibial tray overhangs the lateral chamfer wall of the proximal tibia.

According to additional features, the method can include disposing bone cement between the medial chamfer wall and the medial connecting surface and also between the lateral chamfer wall and the lateral connecting surface.

According to other features, the method can further comprise preparing an anterior cut into the proximal tibia. The anterior cut can create an anterior chamfer wall on the proximal tibia.

According to additional features, the method can include fixing a tibial cut block relative to the proximal tibia based on the determination. A vertical cut guide can be slidably translated along a slot defined in the tibial cut block until a desired medial-lateral position relative to the proximal tibia has been attained. The vertical cut guide can be fixed relative to the tibial cut block based on attaining the desired medial-lateral position.

In one example, preparing the anterior cut can comprise positioning a Rongeur tool having cutting tips that define obtuse angles relative to corresponding cutting arms at a position relative to an anterior portion of the tibia. The cutting arms can be actuated. The cutting tips can cut the bone and prepare the anterior cut.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1-45 illustrate tibial preparation according to one example of the present teachings.

FIG. 1 is a perspective view of an exemplary 4-in-1 cutting block shown with a selectively attachable ACL protector.

FIG. 2 is a perspective view of the 4-in-1 block of FIG. 1.

FIG. 3 is an anterior view of an exemplary tibia shown prior to performing tibial preparation.

FIG. 4 is an anterior view of the tibia of FIG. 3 and shown subsequent to the tibial preparation.

FIG. 5 is an anterior perspective view of the tibia shown with an extramedullary tibial resection guide attached thereto.

FIG. 6 is an anterior perspective view of the proximal tibia and shown with a tibial resection block coupled to the extramedullary tibial resection guide and located against the proximal tibia.

FIG. 7 is a medial perspective view of the proximal tibia of FIG. 8 shown with a terminal end of a modular stylus engaged to the lowest point of the medial tibial plateau.

FIG. 8 is an anterior perspective view of the proximal tibia shown with the modular stylus positioned with a terminal end of the modular stylus engaged to the lowest point of the medial tibial plateau.

FIG. 9 is an anterior perspective view of the proximal tibia of FIG. 8 shown with the tibial resection block coupled with a modular stylus being adjusted to a desired location.

FIG. 10 is an anterior perspective view of the proximal tibia shown with a vertical cut guide coupled to the tibial resection block in line with an ACL and tibial island.

FIG. 11 is an anterior view of the tibia of FIG. 10 and shown with the vertical cut guide coupled to the tibial resection block in a locked position.

FIG. 12 is an anterior view of the proximal tibia shown subsequent to performing a pair of vertical cuts that will form lateral and medial sides of an ACL island made while referencing the vertical cut guide.

FIG. 12A is a cross-sectional view taken along lines 12A-12A of FIG. 12.

FIGS. 12B-12C are an anterior perspective view and cross-sectional view, respectively, of an alternate vertical cut guide.

FIG. 13 is a superior view of the proximal tibia shown with a pre-trial spacer located atop of the lateral plateau to verify the height of tibial bone that was resected.

FIG. 14 is an anterior perspective view of the proximal tibia and pre-trial spacer shown in FIG. 13.

FIG. 15 is a superior view of the proximal tibia shown with a Rongeur tool initially located for resection of the anterior portion of the tibia.

FIG. 16 is a close-up view of the anterior portion of the ACL island of FIG. 15.

FIG. 17 is an anterior perspective view of the proximal tibia of FIG. 15.

FIG. 18 is an anterior perspective view of the tibia of FIG. 17 and shown subsequent to resection of the anterior island and using a rasp to clean up the surface surrounding the ACL island.

FIG. 19 is an anterior perspective view of the proximal tibia shown with a tibial plateau angle gage disposed thereon.

FIG. 20 is a close-up view of a scale of the tibial plateau angle gage of FIG. 19.

FIG. 21 is a perspective view of a spacer tool used to verify a medial and lateral gap.

FIG. 22 is a superior view of the proximal tibia shown using an optional anterior/posterior sizer to verify tibia size.

FIG. 23 is a close-up view of a scale of the sizer shown in FIG. 22.

FIG. 24 is a perspective view of the proximal tibia and shown with a tibial template and anterior/posterior sizer disposed thereon used to verify size, rotation and slope.

FIG. 25 is a lateral view of the proximal tibia shown with the tibial template and anterior/posterior sizer of FIG. 24 disposed thereon.

FIG. 26 is an anterior perspective view of the proximal tibia and shown with the tibial template placed thereon and shown with a drill aligned for receipt by a medial anterior grill guide on the tibial template.

FIG. 27 is an exploded front perspective view of a tibial mask and tibial template.

FIG. 28 is an anterior perspective view of the proximal tibia and shown with a toothbrush keel blade aligned for receipt into a medial passage provided in the tibial template.

FIG. 29 is an anterior view of the proximal tibia of FIG. 28 and shown with the toothbrush keel blade received by the medial passage of the tibial template during formation of a medial groove in the tibia.

FIG. 30 is a front perspective view of a tibial tray trial and tibial tray trial insert constructed in accordance to one example of the present teachings.

FIG. 31 is a front perspective view of the tibial tray trial and tibial tray trial insert shown in an assembled position.

FIG. 32 is an anterior perspective view of the prepared proximal tibia shown with the tibial tray trial and tibial tray trial insert located thereon.

FIG. 33 is a medial perspective view of the proximal tibia and shown with a tibia bearing trial handle and tibial impactor coupled to the tibial tray trial.

FIG. 34 is an anterior perspective view of the proximal tibia of FIG. 33 and bearing trial handle tool.

FIG. 35 is an anterior perspective view of the proximal tibia of FIG. 34 shown with the bearing trial handle tool positioning a bearing onto the tibial tray.

FIG. 36 is a front perspective view of the proximal tibial of FIG. 35 shown with a medial and lateral bearing coupled to the tibial tray.

FIG. 37 is a front perspective view of the tibial tray of FIG. 36 shown with a femoral trial used to check range of motion.

FIG. 38 is an exploded anterior perspective view of an exemplary proximal tibia prepared for receipt of a corresponding tibial tray according to one example of the present teachings.

FIG. 39 is an anterior perspective view of the tibial tray implanted onto the proximal tibia of FIG. 38.

FIG. 40 is a cross-sectional view taken along lined 40-40 of FIG. 39.

FIG. 41 is an exploded anterior perspective view of another proximal tibia prepared for receipt of a tibial tray constructed in accordance to other features of the present disclosure.

FIG. 42 is an inferior perspective view of the tibial tray of FIG. 41.

FIG. 43 is a cross-sectional view taken along lines 43-43 of FIG. 42.

FIG. 44 is an anterior view of an exemplary cut guide used to prepare the medial and lateral chamfer walls of the ACL island according to one example of the present disclosure.

FIG. 45 is an anterior perspective view of the proximal tibia shown in FIG. 41 illustrating an exemplary Rongeur tool preparing the anterior chamfer wall.

Corresponding reference numerals indicate corresponding parts throughout the several views of the drawings.

### DETAILED DESCRIPTION

The following description will focus on preparation of a left knee for receipt of a bi-cruciate knee implant. In this regard, the following description will be directed toward various methods and techniques using instrumentation for preparing a left knee using a bi-cruciate knee system. It will be appreciated however, that the same may be adapted for use with a right knee.

While the intended focus of the instant application will be directed specifically to preparation of the tibia and related implants, a brief description of an exemplary preparation of a left femur will be described. In order to assess bone stock, potential ligament instability and the anatomical axis, a standing anterior/posterior x-ray may be used. In some examples, a 36 inch long standing anterior/posterior x-ray may be used. Initially, the angle between the anatomic and mechanical axis may be determined while assuring that the distal femoral cut is perpendicular to the mechanical axis. At this time, the femoral component size may be estimated pre-operatively by using lateral view x-rays and radio graphic templates. The appropriate size femoral component may be confirmed intra-operatively.

An intramedullary (IM) drill may be used to penetrate the intracondylar notch and dense cancellous bone of the distal femur to a depth of approximately 1.5-2 inches (3.5-5 centimeters). A 0.375 inch drill may be used to penetrate the distal femur. The canal entry location may be placed one centimeter above the insertion of the posterior cruciate ligament and slightly medial in the intracondylar notch. The appropriate left or right valgus wing may be chosen and slid onto the IM rod. The IM rod may be introduced into the femoral canal to de-pressurize the canal. The valgus wing may be slid until it rests against the medial distal condyle. The Slidex® Distal Resection Block and cut block adapter are both slid into the anterior holes of the valgus wing until the Slidex® Distal Resection Block contacts the anterior cortex of the femur.

To confirm the valgus angle, the alignment handle can be inserted into the cut block adapter and a ¼ inch alignment rod can be inserted and extended to the center of the femoral head. The Slidex® Distal Resection Block can then be pinned into place using ⅛ inch quick release drill pins in the most proximal pin holes of the block. The valgus wing can then be removed by removing the IM rod and pulling the valgus wing and cut block adapter distally away from the distal resection block leaving the Slidex® Distal Resection Block in place. Two resection slots of 0 or +3 mm are available for the distal resection. The 0 mm slot will resect 9 mm from the most prominent part of the medial distal condyle. If additional distal resection is required, the +3 mm slot will resect 12 mm. If additional distal resection is required beyond the +3 mm slot, the resection guide can be shifted proximally by utilizing the +2 or +4 mm ⅛ inch pin holes. A 0.054 inch saw blade can be used to complete the distal resection through the selected slot. The resected distal femur can be checked by using a flat instrument. The bone surface may be re-cut or filed as necessary to ensure proper resection. For additional stability, the femoral block handle can be utilized.

An exemplary method of femoral sizing will now be described. Initially, the adjustable anterior/posterior sizer may be placed against the resected distal surface with the feet in contact with the posterior condyles of the femur. In a first option, fixed rotation feet may be used. In another option, adjustable rotation feet may be used. An adjustable dial can be used with the anterior/posterior sizer. The adjustable rotation feet are available in left and right varieties with the ability to set an external rotation from 0 to 10 degrees. In one example, it is recommended that an initial setting of 3 degrees of rotation be utilized. The femoral component size can now be read from the central scale. If the size indicated is in between standard sizing or a larger flexion gap is desired, a choice may be made to choose the smaller size and shift the femoral 4-in-1 block placement anteriorly. In order to shift the component anteriorly, a screw mechanism in the central portion of the sizer is turned which raises the level of drill holes in one millimeter increments. A scale is located on the sizer to indicate how far the component will be anteriorly shifted. If medial/lateral width is a concern, the appropriately sized medial/lateral width checker can be inserted into the anterior/posterior sizer to further evaluate the proper size of the femur. Next, two 4-in-1 cutting block location holes are drilled utilizing a ⅛ inch drill pin. In one example, the final medial/lateral position of the femoral component is not determined during this step, but is addressed later in the technique.

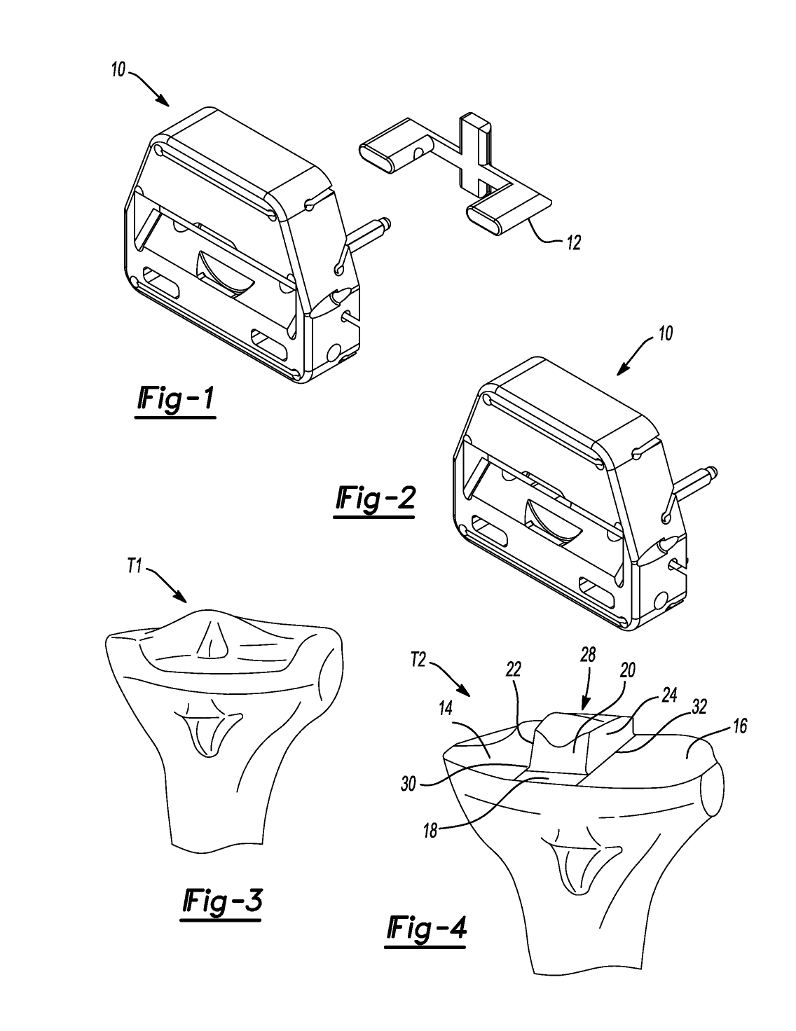

With initial reference now to FIGS. 1 and 2, initial preparation of the distal femur using a 4-in-1 block 10 according to the present teachings will be described. At the outset, a surgeon may choose the desired 4-in-1 block 10 that matches the selected size on the anterior/posterior sizer and place it into the ⅛ inch holes drilled into the distal femur. A 0.054 inch feeler blade can be used to determine the amount of anterior bone resection. If the feeler blade indicates a probability of notching, an anterior/posterior femoral shift block may be used to adjust the cut block holes anteriorly in one millimeter increments. Notably, moving the block anteriorly will resect additional posterior condylar bone. ⅛ inch pins can be placed in the side holes provided on the femoral 4-in-1 block 10. The anterior/posterior block must be sitting flush against the distal femur at this point. An ACL protector 12 may be secured into place relative to the 4-in-1 block 10. The ACL protector 12 can be used to block the blade from inadvertently cutting the ACL. Once the position of the 4-in-1 block 10 is satisfactory, a surgeon can resect the anterior and posterior bone, and the anterior and posterior chamfers using a 0.054 inch saw blade. Again, care must be taken not to cut the ACL while making the posterior and posterior chamfer boney resections.

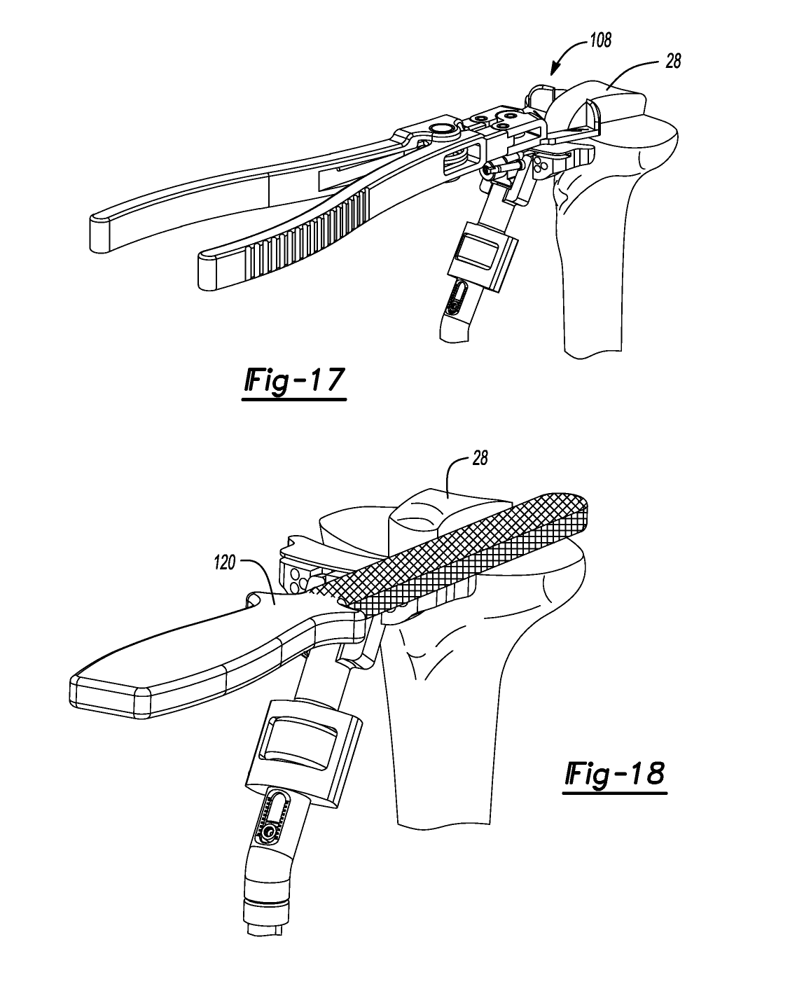

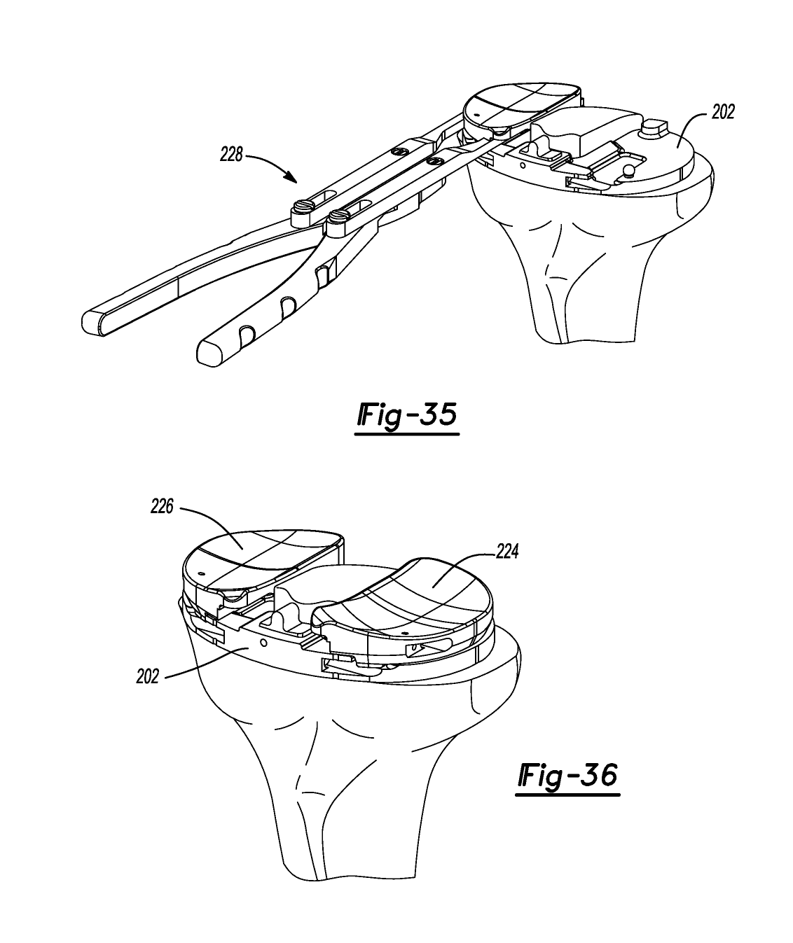

With reference now to FIGS. 3-37, preparation of a proximal tibia for a bi-cruciate knee system according to a first example will be described. FIG. 3 illustrates a tibia T1 prior to performing the instant surgical technique. FIG. 4 illustrates a tibia T2 subsequent to performing the tibial technique according to the present teachings. Of note, the tibia T2 includes a medial plateau 14, lateral plateau 16, anterior plateau 18, anterior chamfer wall 20, medial vertical wall 22, and lateral vertical wall 24. The anterior chamfer wall 20, the medial vertical wall 22, and the lateral vertical wall 24 can collectively cooperate to form an ACL island 28. A radius 30 is formed at a transition between the medial plateau 14 and the medial vertical wall 22. Similarly, a radius 32 is formed at a transition between the lateral plateau 16 and the lateral vertical wall 24.

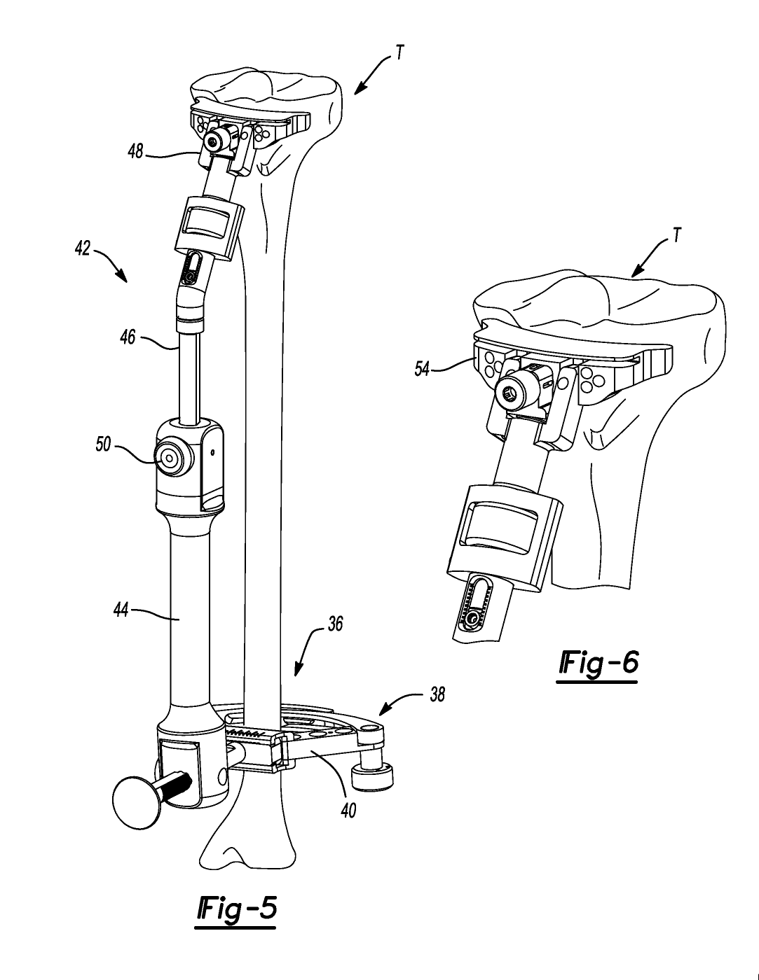

With reference now to FIGS. 5-29, resection of the tibia T will be described. With the knee flexed, spring loaded arms 36 and 38 of an ankle clamp 40 are located around the distal tibia T just around the malleoli. The ankle clamp 40 can generally be attached to an extramedullary tibial resection guide 42. The extramedullary tibial resection guide 42 can further comprise a handle portion 44, a telescoping rod portion 46, and a resection block connecting portion 48. A button 50 can be provided on the extramedullary tibial resection guide 42 that can control telescoping action of the rod portion 46 generally from the handle portion 44.

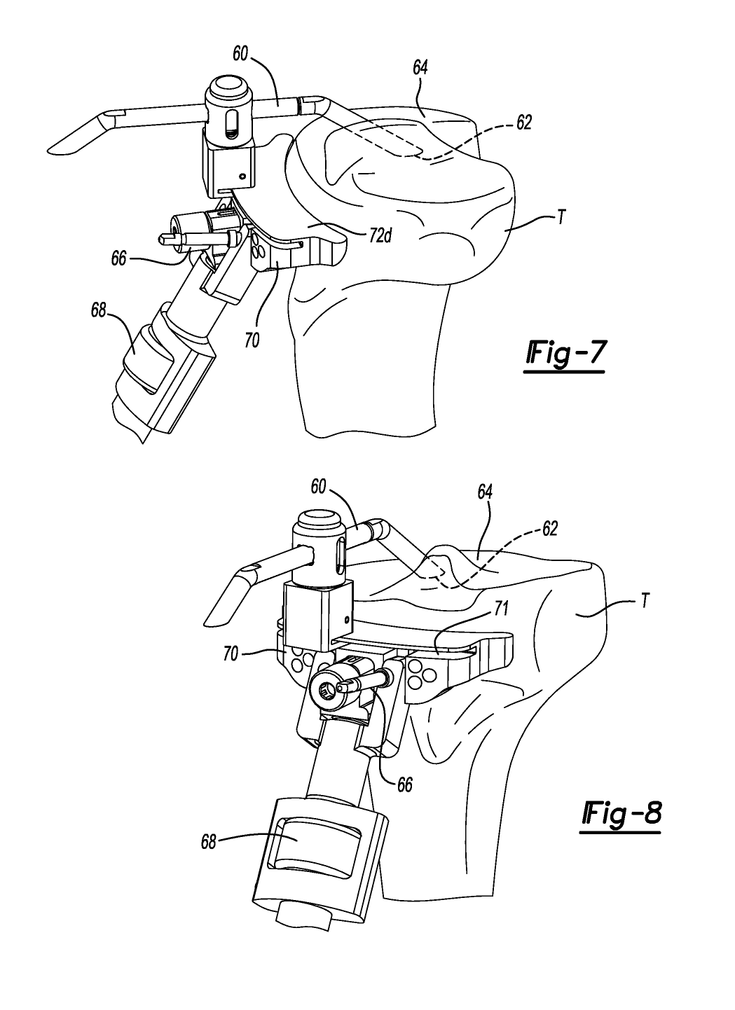

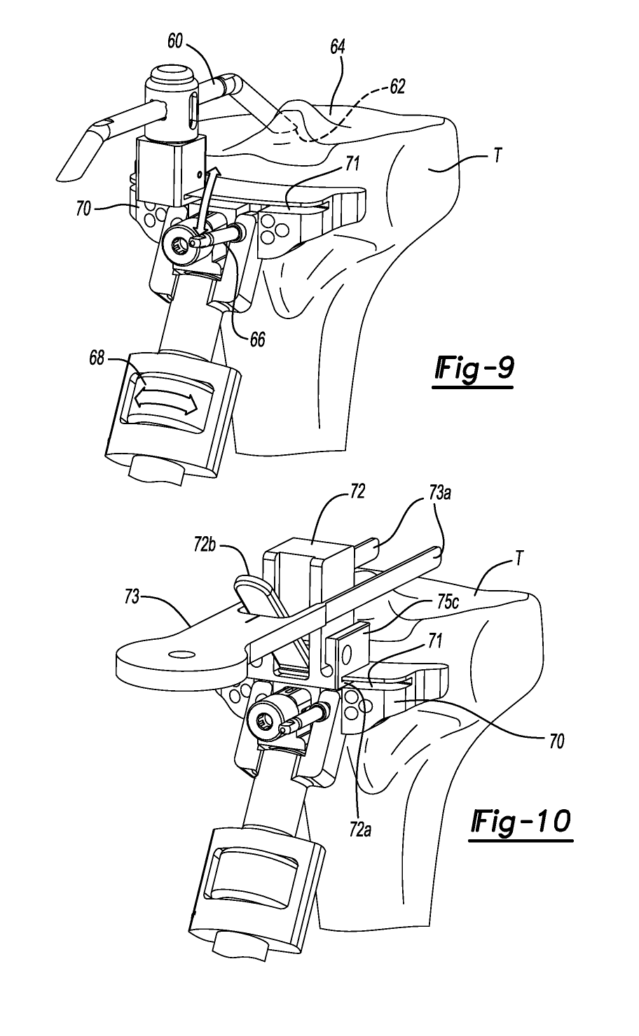

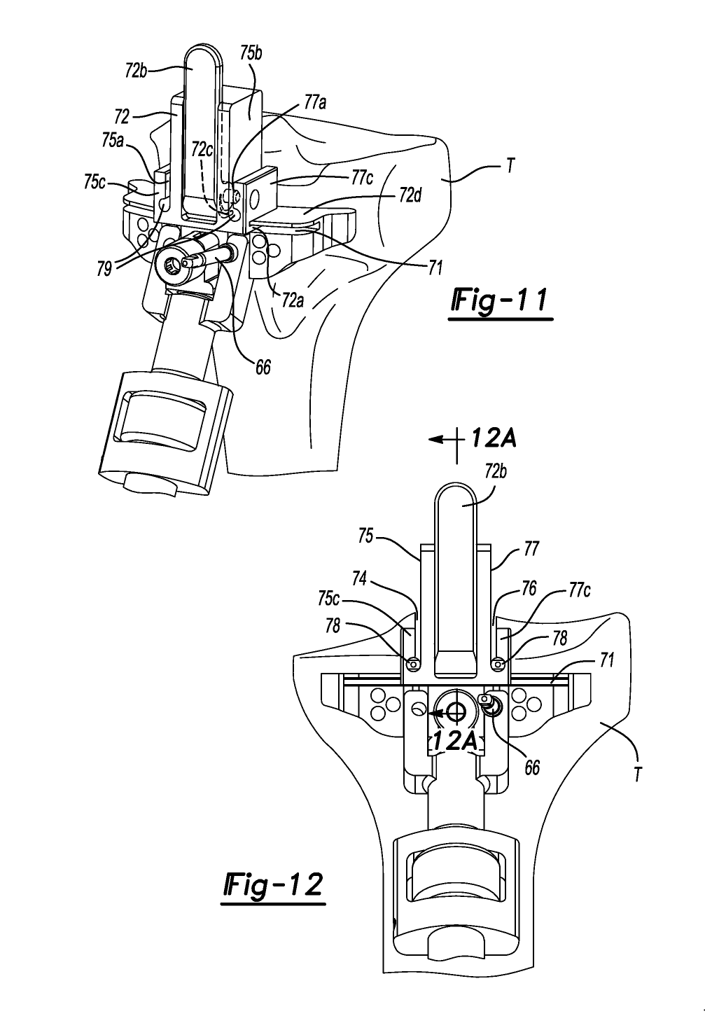

At this point, a tibial resection block 54 (FIG. 6) can be placed against the proximal tibia T. Returning now to FIG. 5, from the sagittal view, the side of the extramedullary tibial resection guide 42 is adjusted such that it is generally parallel with the shaft of the tibia T. The tibial resection block is set at 4 degrees of slope (other measurements may be used) when attached to the extramedullary guide. Once adjustment of the resector axis is correct in the medial/lateral view, the resection block connecting portion 48 is rotated until the shaft of the resector is just medial to the tibial tubercle. Using a stylus 60 (FIGS. 7 and 8), the extramedullary tibial resection guide 42 is adjusted such that a terminal end 62 of the stylus 60 is engaged to a lowest point of the medial tibial plateau 64. Using a ⅛ inch pin 66, the extramedullary tibial resection guide 42 is secured to the tibia T. A dial 68 may be used to fine tune the resection level prior to making any cut (FIG. 9).

Of note, the stylus 60 is set for a 4 mm resection. Prior to pinning the extramedullary tibial resection guide 42 in place, make sure to allow for adjustability of the height of a tibial resection cut block 70. The tibial resection cut block 70 can define a horizontal slot 71. Once the resection level is set, the stylus 60 can be removed. A vertical cut guide 72 can then be attached to the tibial resection cut block 70 (FIG. 10).

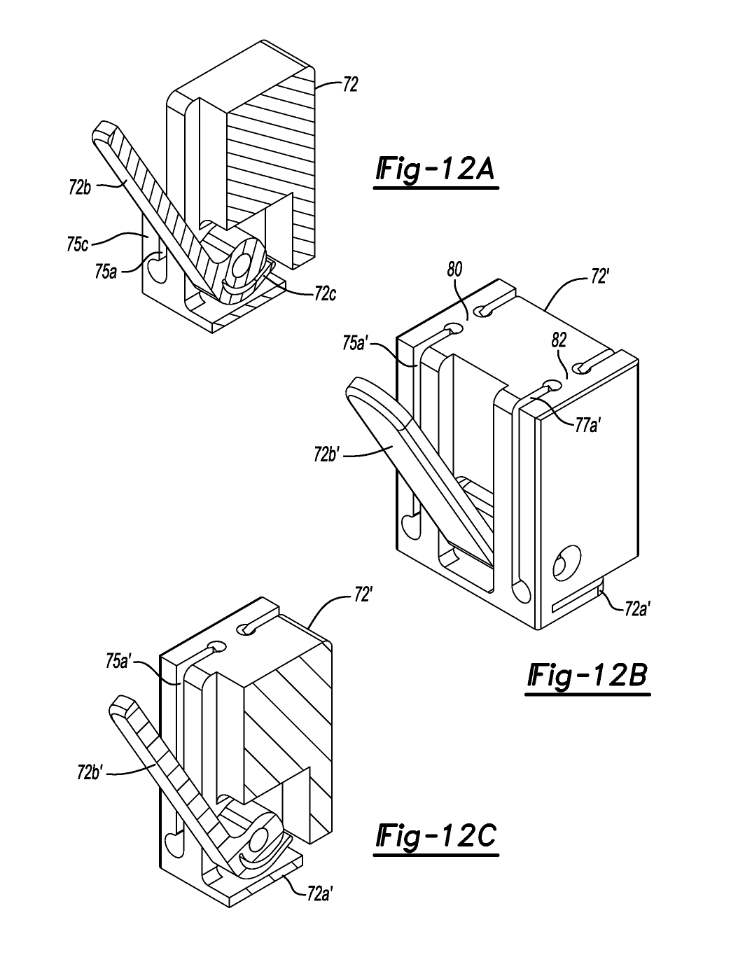

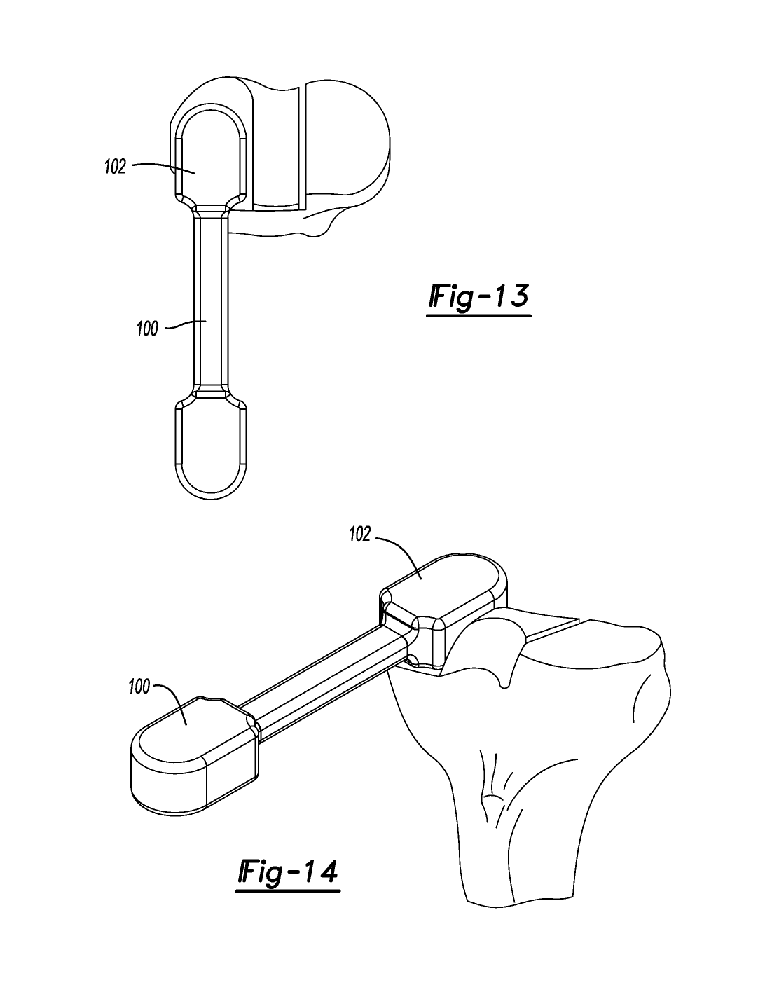

The vertical cut guide 72 can then be adjusted to an appropriate position (in a medial/lateral direction along the slot 71) to make the desired vertical cuts. Specifically, a tongue 72a extending from the vertical cut guide 72 can slide along the slot 71. An alignment guide 73 can be used to aid in the positioning of the vertical cut guide 72. The alignment guide 73 generally includes a pair of parallel and elongated arms 73a that slidably locate on opposite sides of the vertical cut guide 72. Of note, the vertical cuts will determine the final tibial component rotation. It is important to leave equal amounts of bone on the medial and lateral aspect of the ACL fibers. At this point, the vertical cut guide 72 can be clamped in place by rotating a locking arm 72b from an unlocked position shown in FIG. 10 to a locked position shown in FIGS. 11 and 12. In one example, the locking arm 72b can have a finger 72c that rotates into fixed engagement with an upper surface 72d of the cut block 70. With a reciprocating saw, a vertical medial cut 74 can be prepared while passing a saw through a medial slot 75a defined between a main body 75b of the vertical cut guide 72 and a medial arm 75c. The vertical medial cut 74 may be prepared while referencing a medial surface 75 of the vertical cut guide 72. It will be appreciated that the vertical medial cut 74 may be prepared while concurrently referencing the medial arm 75c. After the vertical medial cut 74 has been prepared, the vertical lateral cut may be made. The vertical lateral cut 76 can be prepared while passing a saw through a lateral slot 77a defined between the main body 75b of the vertical cut guide 72 and a lateral arm 77c. The vertical lateral cut 76 may be prepared while referencing a lateral surface 77 of the vertical cut guide 72. It will be appreciated that the vertical lateral cut 76 may be prepared while concurrently referencing the lateral arm 77c. Headless vertical pins 78 can be located through partial bores 79 (FIGS. 11 and 12) provided in the vertical cut guide 72 driven into the anterior tibia T. The vertical medial cut 74 and the vertical lateral cut 76 can both be prepared using a saw blade having teeth or cutting structure consistent for forming the radius cuts 30 and 32 identified in FIG. 4. Notably, by incorporating a radius at this transition, the bone at the transition between the respective medial and lateral plateaus 14, 16 and ACL island 28 (FIG. 4) can be stronger as compared to a transverse, 90 degree intersecting cut. Next, the vertical cut guide 72 is removed from the headless vertical pins 78. The medial side of the tibia T may then be horizontally resected.

With reference to FIG. 12A, a cross-sectional view of the cut guide 72 is shown. FIGS. 12B and 12C show an alternate vertical cut guide 72′. Unless otherwise described herein, the cut guide 72′ incorporates similar features as the cut guide 72 that are identified with like reference numerals having a prime suffix. The cut guide 72′ provides a captured vertical medial slot 75a′ and a captured vertical lateral slot 77a′. Specifically, an upper medial wall 80 and an upper lateral wall 82 close the respective vertical medial slot 75a′ and the vertical lateral slot 77a′. The upper medial and lateral walls 80 and 82 can assist in maintaining a saw blade within the respective medial and lateral slots 75a′ and 77a′.

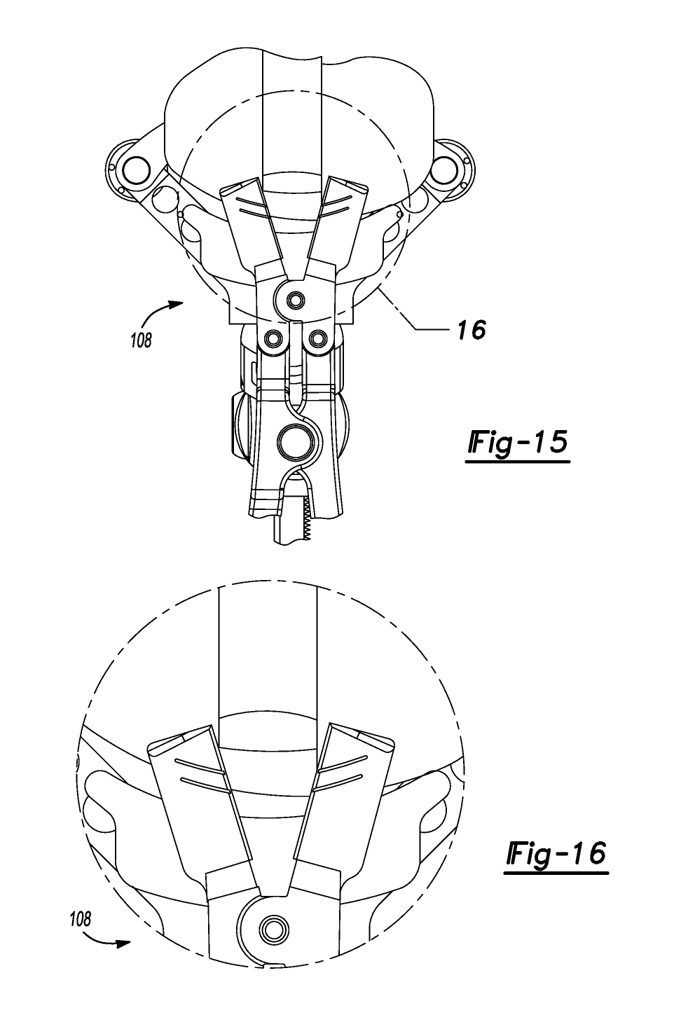

At this point, the medial side gap may be verified in extension using an 8/9 mm spacer block 100 (FIGS. 13-14). If the 9 mm spacer portion 102 is too tight, additional tibial bone will need to be removed. This can be done by simply dialing the resection block down 1 mm. Once the medial side extension gap is adequate, the lateral side of the tibia T is horizontally resected with the headless vertical pins 78 left in place. The headless vertical pins 78 protect against undercutting the ACL island 28.

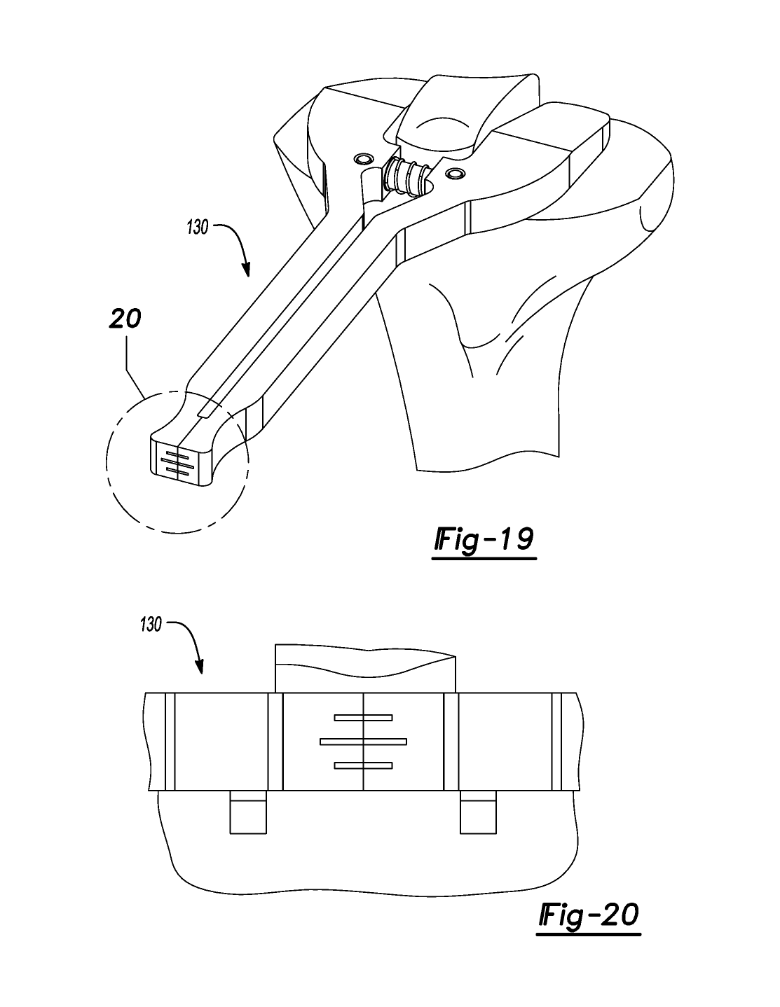

As illustrated in FIGS. 15-17, a Rongeur tool 108 can be used to remove the anterior bone making sure to round the corners of the anterior island. Next, an ACL island rasp 120 (FIG. 18) is used to clean the resected tibia T to ensure that there are no rough edges around the ACL island 28 and respective medial and lateral plateaus 14 and 16. Using the tibial plateau angle gage 130 (FIG. 19), the tibial slope cuts are verified to have an equal amount of slope. This will be important for the tibial base plate to be secured properly, and for the proper wear and function of the system.

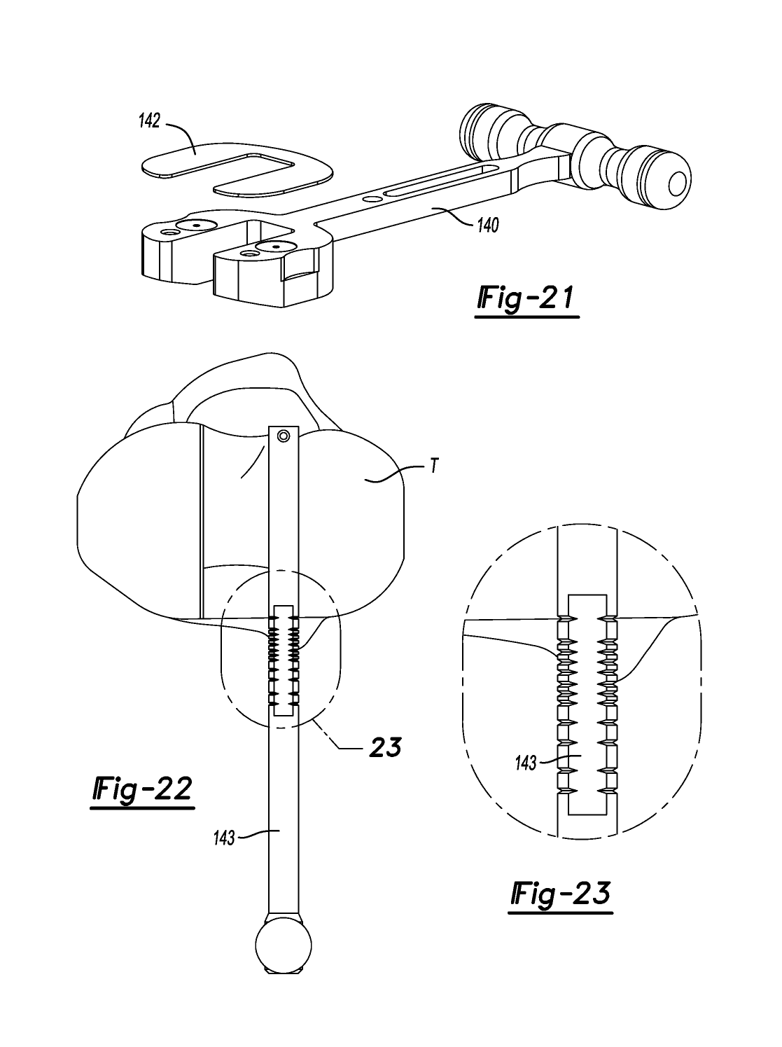

Turning now to FIG. 21, tibial sizing for an intact and functional ACL will be described. The medial and lateral gaps are verified using a spacer tool 140. A series of 1 mm spacers 142 may be magnetically coupled as needed. Rotation and slope may also be verified. Optionally, the tibia T may be sized with an anterior/posterior sizer 143 (FIGS. 22 and 23).

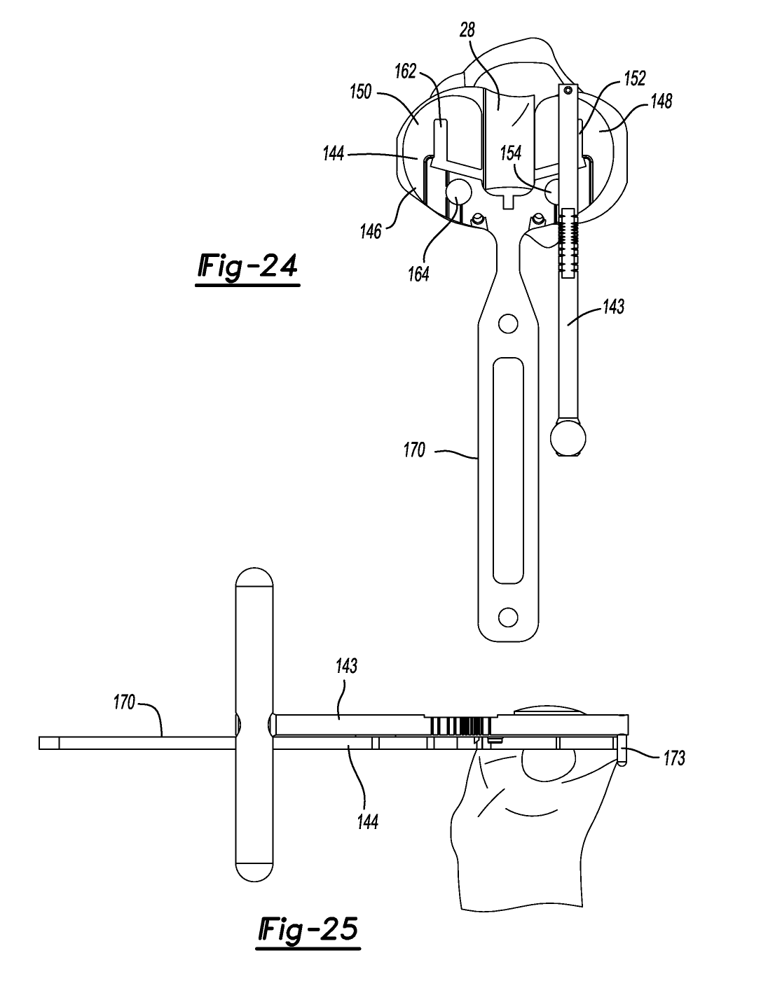

The tibia T may then be sized with a tibial template 144 (FIGS. 24-25). The tibial template 144 generally comprises a U-shaped body portion 146 having a lateral side 148, and a medial side 150. A lateral passage 152 and a lateral anterior drill guide 154 can be provided on the lateral side 148. Similarly, a medial passage 162 and medial anterior drill guide 164 can be provided on the medial side 150. Because rotation is determined by the position of the ACL island 28, it is important to check for accurate rotation. Base rotation can be made relative to the tibial tubercle and the malleolar axis. At this point, an extramedullary alignment check can be made by placing a ¼ inch alignment rod through a handle 170 of the tibial template 144. Slight external rotation is preferred to optimize patellofemoral tracking. Once the final rotation has been determined, the position can be marked by extending anterior marks of the tibial template 144 onto the anterior tibia such as by electrocautery. A locator pin 173 extending from the anterior/posterior sizer 143 can be located around the posterior edge of the tibia T. Extra caution should be used to avoid internal rotation of the tibial template 144 due to the presence of lateral soft tissue.

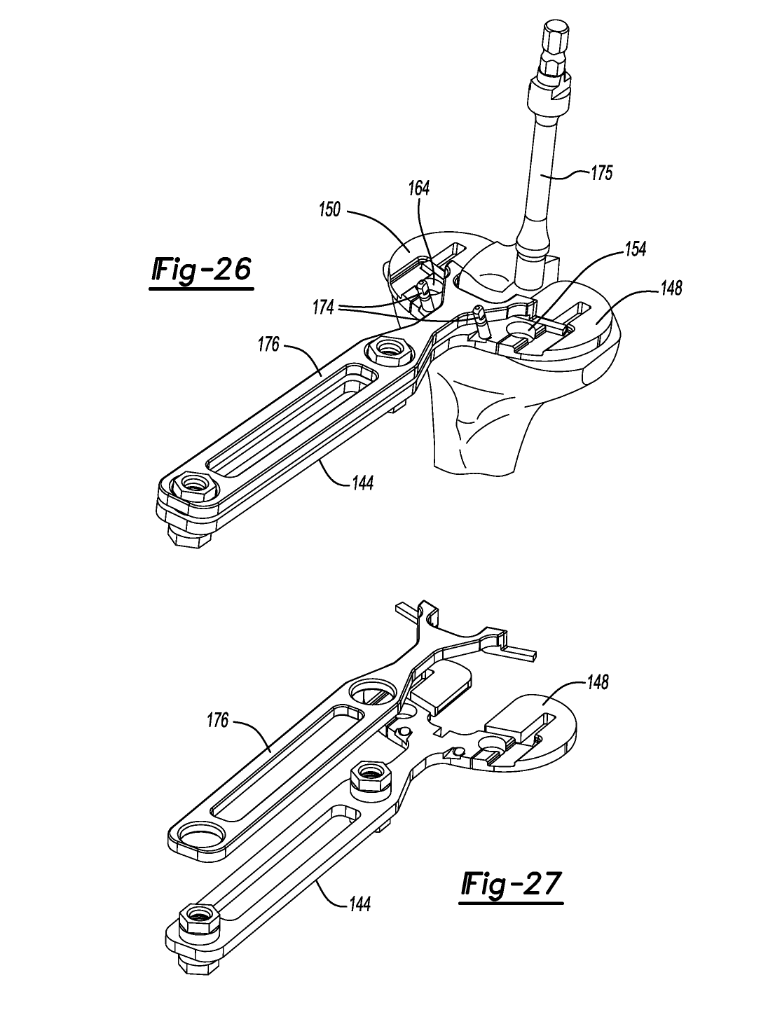

Tibial preparation for an intact and functional ACL will now be described. With the tibial template 144 in proper position (FIG. 26), such as by way of pins 174, a drill 175 can be used to prepare an anterior hole while referencing the lateral anterior drill guide 154. A tibial mask 176 may be coupled to the tibial template 144. In one example, a ⅛ inch drill 175 may be used (FIG. 26). Next, another anterior hole can be drilled with the drill 175 while referencing the medial anterior drill guide 164.

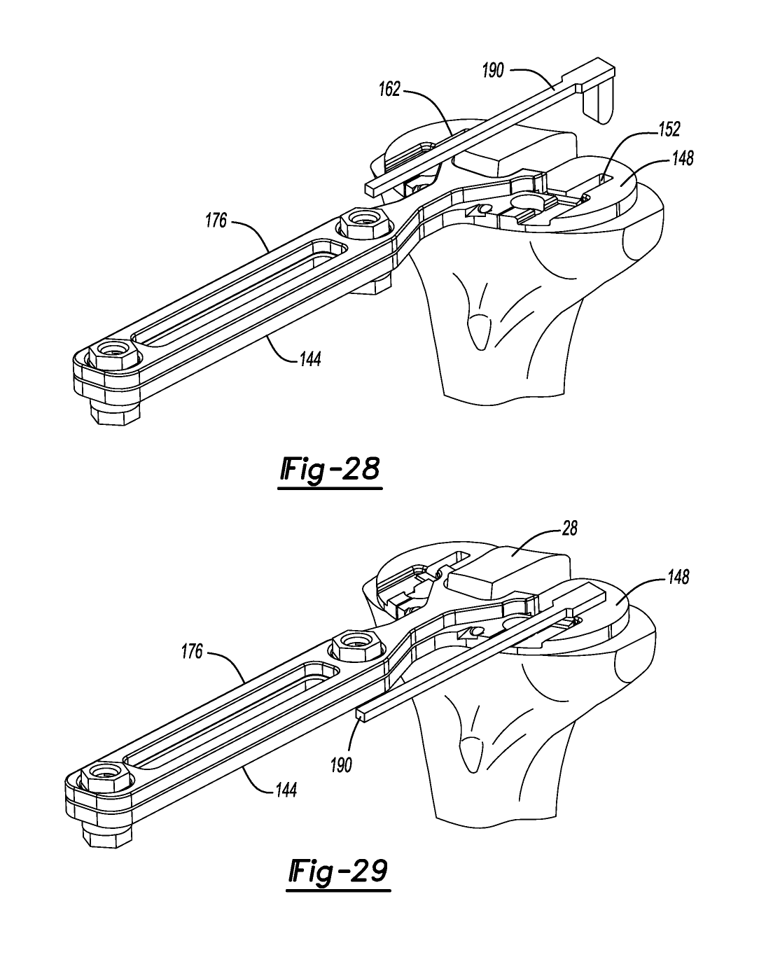

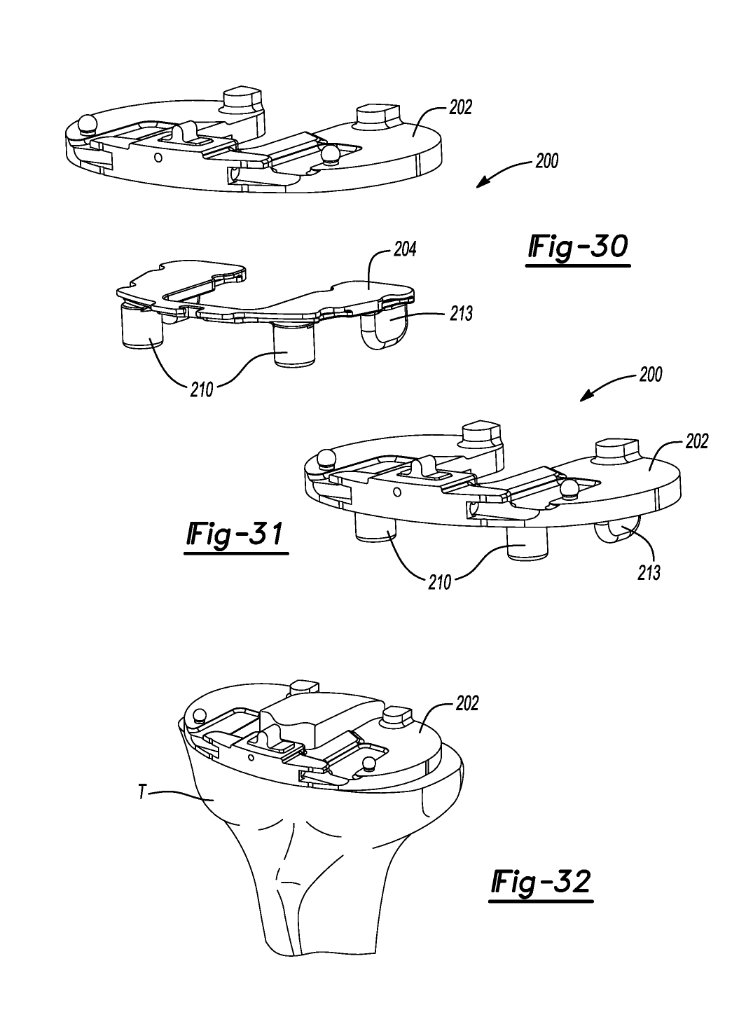

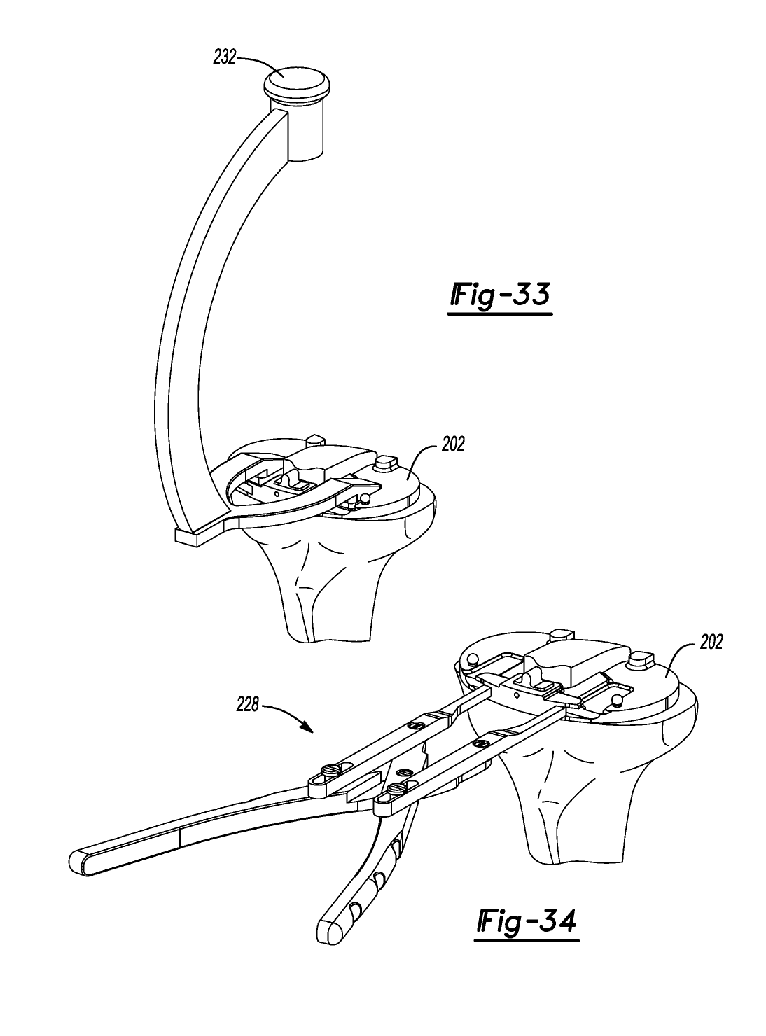

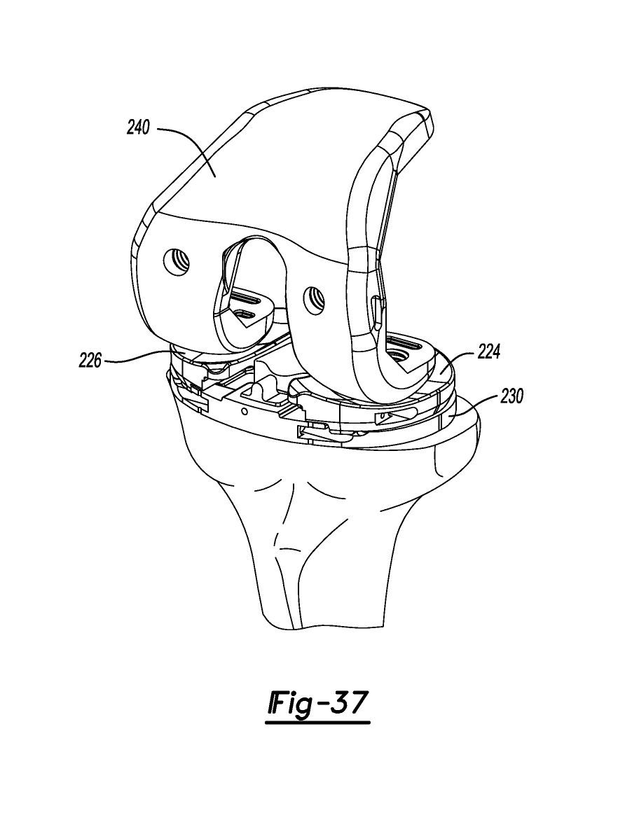

With the tibial template 144 secured in place, a toothbrush keel blade 190 can be used to prepare both the medial and lateral tibia for the keeled base plate. Specifically, the toothbrush keel blade 190 can be inserted through the lateral passage 152 and the medial passage 162 (FIGS. 28 and 29). While the tibia T is being prepared, the tibial trial assembly 200 (FIGS. 30 and 31) can be prepared. The tibial trial assembly 200 can include a tibial tray trial 202 and tibial tray trial insert 204. Once tibial preparation is complete, the tibial template 144 can be removed from the proximal tibia. The tibial tray trial 202 can have multiple versions that provide various dimensions. Similarly, the tibial tray trial insert 204 can also provide various dimensions suitable for the needs of a particular patient. Of note, the tibial tray trial insert 204 includes pegs 210 and keels 213. The pegs 210 have a spacing that corresponds to the passages made earlier with the drill 175. Similarly, the keels 213 have dimensions suitable for insertion into the grooves prepared with the toothbrush keel blade 190. As illustrated in FIG. 33, a tibial tray trial 202 is shown being impacted onto the tibia T using a tibial impactor 232. As illustrated in FIGS. 34-36, a lateral tibial bearing trial 224 and a medial tibial bearing trial 226 can be coupled to the tibial tray trial 202 using a bearing trial handle tool 228 and trialed. Also, the tibial tray trial 202 can be positioned with the bearing trial handle tool 228 (FIG. 34). As shown in FIG. 37, a femoral trial 240 can be used to verify range of motion.

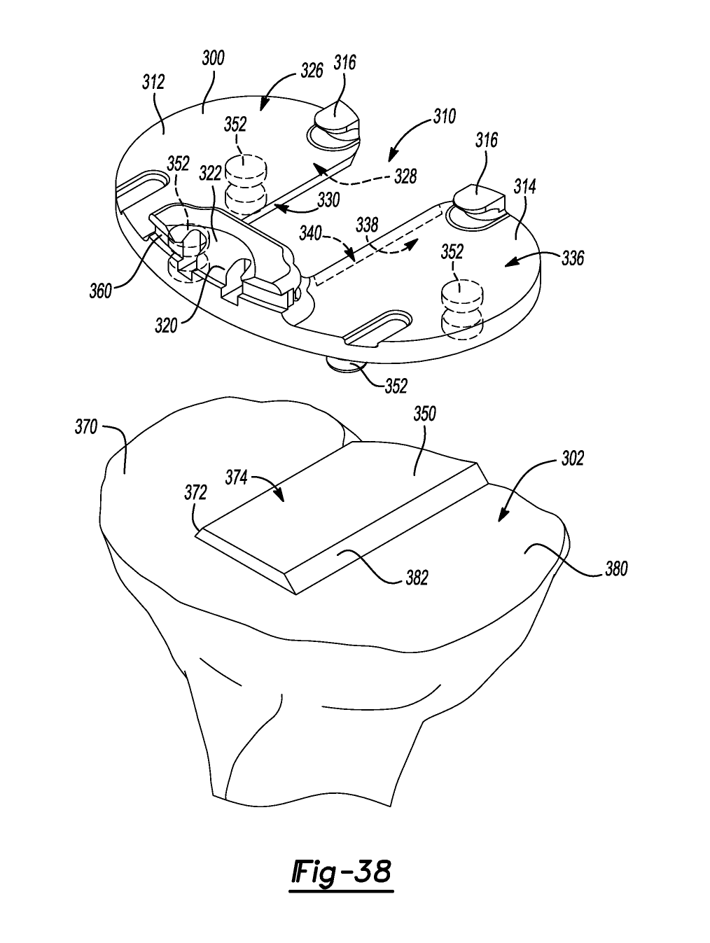

With reference now to FIGS. 38-45, various tibial trays and related surgical methods for preparing an ACL island according to additional features of the present disclosure will be described. In general, the tibial trays and corresponding surgical preparation techniques provide a configuration where portions of the tibial tray will overhang corresponding bone of the ACL island. In this regard, and as will be described more fully herein, the tibial tray may provide various surfaces that extend in part above (in a superior direction) corresponding portions of the ACL island. In this regard, the configuration of the tibial tray may inhibit fracture or dislocation of the ACL island from the proximal tibia. In some examples discussed herein, a flowable adhesive such as bone cement may be disposed between the corresponding features of the tibial tray and the ACL island.

With particular reference now to FIG. 38, a tibial tray constructed in accordance to one example of the present disclosure is shown and generally identified at reference numeral 300. As will be discussed, the tibial tray 300 is configured for implanting onto a correspondingly prepared proximal tibia 302. The tibial tray 300, as with those described above, can be generally U-shaped and provides a slot 310 that can be configured to accommodate and provide clearance for a host ACL and/or PCL or a reconstructed ACL and/or PCL. Other examples of attaching an artificial or natural ACL and/or PCL may be found in “Knee Prosthesis Assembly With Ligament Link”, Ser. Nos. 12/788,966 and 12/788,973 (Attorney Docket Nos. 5490-000809/US and 5490-000809/US/01). The tibial tray 300 can include a medial portion 312, a lateral portion 314, posterior engagement tabs 316, an anterior engagement bridge 320 and an anterior connecting portion 322. In general, the anterior connecting portion 322 connects the medial portion 312 and the lateral portion 314 and cooperates with the medial and lateral portions 312 and 314 to define the slot 310. The medial portion 312 can further include a superior medial bearing opposing surface 326, an inferior medial bone opposing surface 328 and a medial connecting surface 330. The lateral portion 314 can generally include a superior lateral bearing opposing surface 336, an inferior lateral bone opposing surface 338, and a lateral connecting surface 340. As will become appreciated from the following discussion, the medial and lateral connecting surfaces 330 and 340 can be formed on an angle or bevel to create a chamfer surface that can be configured to overhang corresponding bone of an ACL island 350 on the proximal tibia 302. The tibial tray 300 can further include inferior posts 352. Other engaging structure such as keels for example, may additionally or alternatively be provided on the tibial tray 300. The anterior engagement bridge 320 can include a track 360 formed thereon. The anterior engagement bridge 320 can provide an increased thickness to the tibial tray 300 at the connection between the medial and lateral portions 312 and 314, respectively to increase durability.

With continued reference to FIG. 38, the ACL island 350 of the proximal tibia 302 will be further described. In general, the ACL island 350 can be prepared onto the proximal tibia 302 similar to that described above with the following distinctions. A medial cut may be prepared into the proximal tibia 302 having an obtuse angle relative to a medial plateau 370. The medial cut creates a medial chamfer wall 372 between the medial plateau 370 and a superior surface 374 of the ACL island 350. Similarly, a lateral cut is prepared onto the proximal tibia 302. The lateral cut can have an obtuse angle relative to a lateral portion 380 of the proximal tibia. The lateral cut can create a lateral chamfer wall 382 on the proximal tibia 302 between the lateral portion 380 and the superior surface 374 of the ACL island 350.

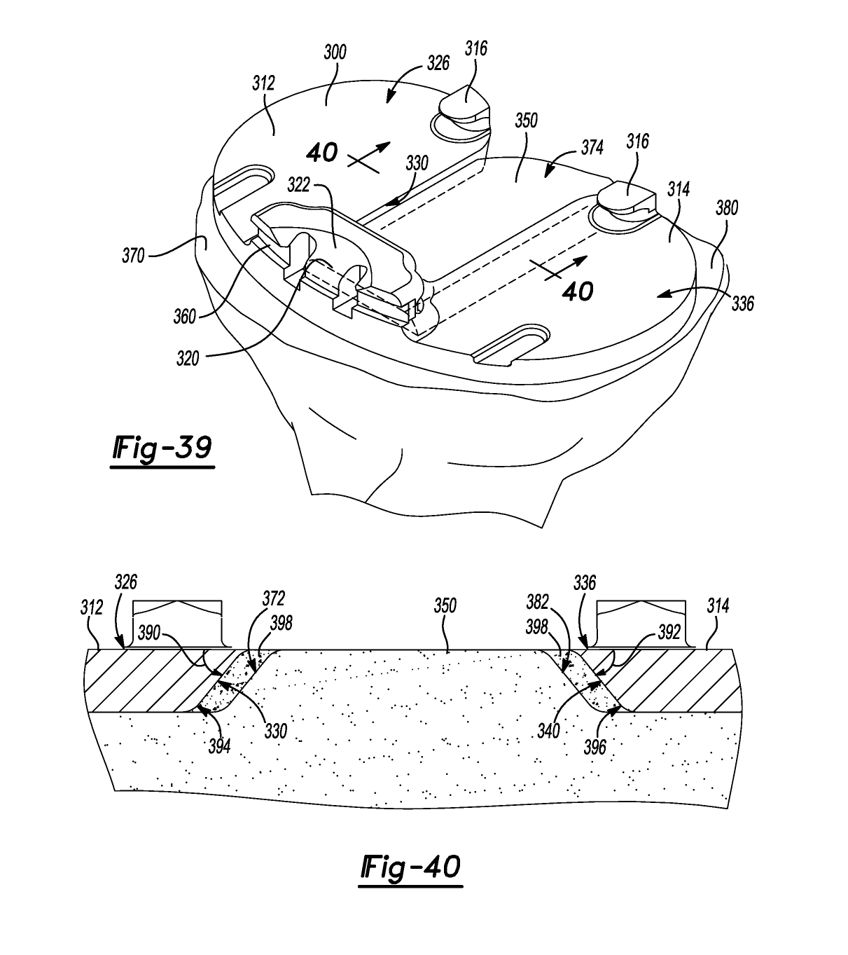

With further reference now to FIGS. 39 and 40, the medial chamfer wall 372 can be prepared in such a manner that the corresponding medial connecting surface 330 on the tibial tray 300 will overhang at least a portion of the ACL island 350. Similarly, the lateral chamfer wall 382 can be prepared in a way such that the lateral connecting surface 340 of the tibial tray 300 will overhang at least a portion of the ACL island 350. As used herein, the term “overhang” is used to denote a geometrical relationship between the medial connecting surface 330 and corresponding medial chamfer wall 372 wherein a vertical line that intersects a portion of the medial chamfer wall will also intersect a portion of the medial connecting surface 330. A similar relationship may be attained between the lateral chamfer wall 382 and the lateral connecting surface 340. In the example shown, the medial connecting surface 330 can define an angle 390 of between 30 degrees and 60 degrees relative to the superior medial bearing opposing surface 326. In the example shown, the angle 390 can be about 45 degrees. Similarly, the lateral connecting surface 340 can define an angle 392 of between 30 degrees and 85 degrees relative to the superior lateral bearing opposing surface 336. In the example shown, the angle 392 can be about 45 degrees. In some examples, the medial connecting surface 330 can provide a medial connecting plane 394. The lateral connecting surface 340 can define a lateral connecting plane 396. The medial connecting plane 394 and the lateral connecting plane 396 can generally converge above the superior medial and lateral surfaces 326 and 336.

In the example shown in FIG. 40, a flowable material such as bone cement 398 can be disposed between the medial connecting surface 330 and the medial chamfer wall 372. Similarly, the bone cement 398 can be disposed between the lateral connecting surface 340 and the lateral chamfer wall 382.

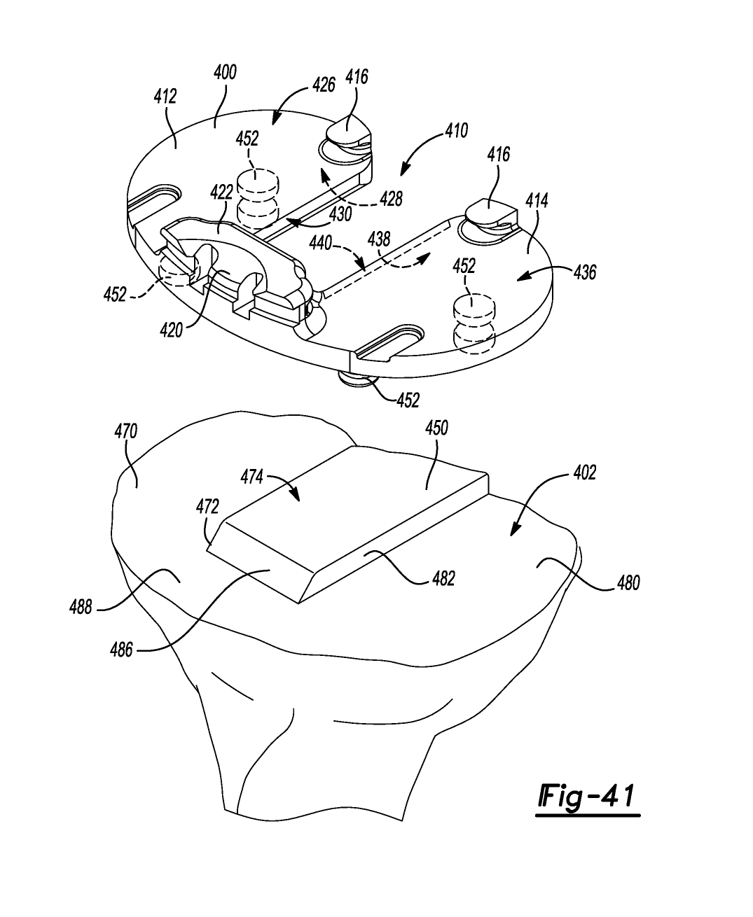

With particular reference now to FIG. 41, a tibial tray constructed in accordance to another example of the present disclosure is shown and generally identified at reference numeral 400. As will be discussed, the tibial tray 400 is configured for implanting onto a correspondingly prepared proximal tibia 402. The tibial tray 400, as with those described above, can be generally U-shaped and provides a slot 410 that can be configured to accommodate and provide clearance for a host ACL and/or PCL or a reconstructed ACL and/or PCL.

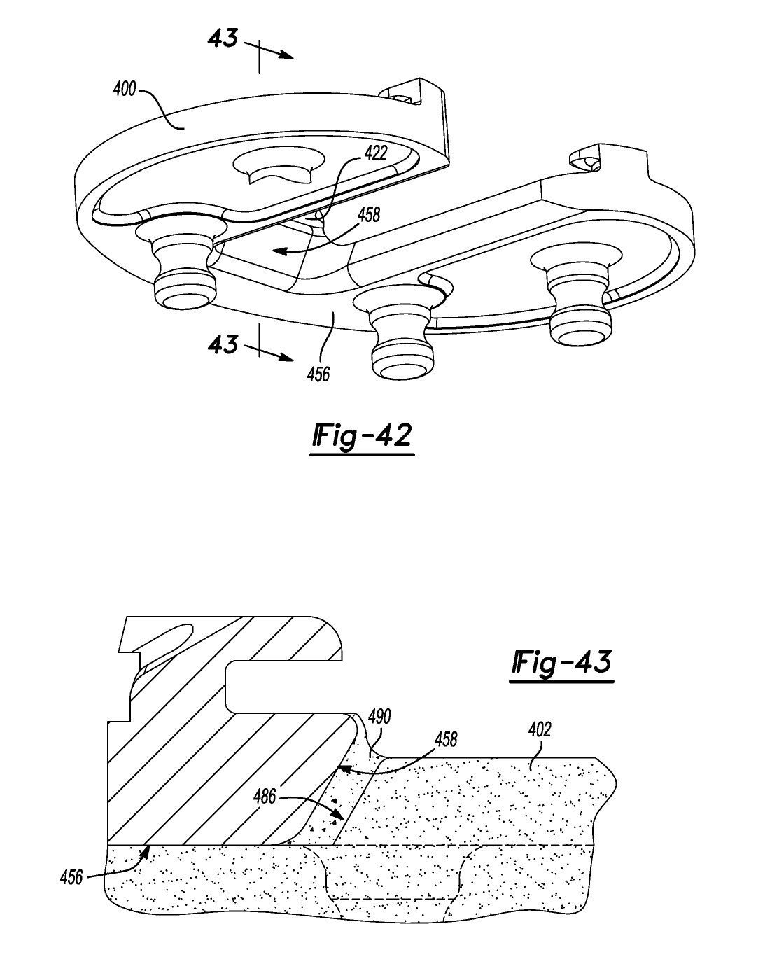

The tibial tray 400 can include a medial portion 412, a lateral portion 414, posterior engagement tabs 416, an anterior engagement bridge 420 and an anterior connecting portion 422. In general, the anterior connecting portion 422 connects the medial portion 412 and the lateral portion 414 and cooperates with the medial and lateral portions 412 and 414 to define the slot 410. The medial portion 412 can further include a superior medial surface 426, an inferior medial surface 428 and a medial connecting surface 430. The lateral portion 414 can generally include a superior lateral surface 436, an inferior lateral surface 438, and a lateral connecting surface 440. The medial and lateral connecting surfaces 430 and 440 can be formed at an angle similar to the medial and lateral connecting surfaces 330 and 340 described above, or alternatively, may be formed generally vertically. The tibial tray 400 can further include inferior posts 452. Other engaging structures such as keels, for example, may additionally or alternatively be provided on the tibial tray 400. The anterior connecting portion 422 can further include an inferior anterior bone opposing surface 456 and an anterior connecting surface 458. The anterior connecting surface 458 can be a chamfered surface that defines an obtuse angle relative to the inferior anterior bone opposing surface. In one example, the anterior connecting surface 458 can extend at an angle between 120 degrees and 150 degrees relative to the inferior anterior bone opposing surface 456 as best shown in FIGS. 42 and 43.

With reference to FIG. 41, the ACL island 450 of the proximal tibia 402 will be further described. In general, the ACL island 450 can be prepared onto the proximal tibia 402 similar to that described above with the following distinctions. A medial cut may be prepared into the proximal tibia 402 having a right angle relative to a medial plateau 470. The medial cut can create a medial connecting wall 472 between the medial plateau 470 and a superior surface 474 of the ACL island 450. It will be appreciated that a transition between the medial connecting wall 472 and the medial plateau may have a radius such as described above. Similarly, a lateral cut is prepared onto the proximal tibia 402. The lateral cut can have a right angle relative to a lateral plateau 480 of the proximal tibia 402. The lateral cut can create a lateral connecting wall 482 on the proximal tibia 402 between the lateral portion 480 and the superior surface 474 of the ACL island 450. Again, it will be appreciated that a radius may be formed at a transition between the lateral connecting wall 482 and the lateral plateau 480. An anterior chamfer wall 486 may be formed at a transition between an anterior plateau 488 and the superior surface 474 of the ACL island 450. The anterior chamfer wall 486 can generally be formed at an obtuse angle relative to the anterior plateau 488. In one example, the chamfer wall can define an angle between 120 and 150 degrees relative to the anterior plateau 488 to complement the angle of the anterior connecting surface 458 on the tibial tray 400.

In an assembled position, the anterior connecting surface 458 on the tibial tray 400 can overhang the anterior chamfer wall 486 of the ACL island 450. In this regard, the overhanging relationship can inhibit the ACL island 450 from breaking away from the proximal tibia. In some examples, bone cement 490 may be disposed between the anterior connecting surface 458 and the anterior chamfer wall 486. It will be appreciated that in some examples, the tibial tray may incorporate the chamfered walls of the medial connecting surface 330 and lateral connecting surface 340 of the tibial tray 300 in addition to the anterior connecting surface 458 of the tibial tray 400. In this regard, the ACL island 450 may be prepared having the corresponding medial chamfer wall 372, lateral chamfer wall 382 (FIG. 38) as well as the anterior chamfer wall 486 (FIG. 41).

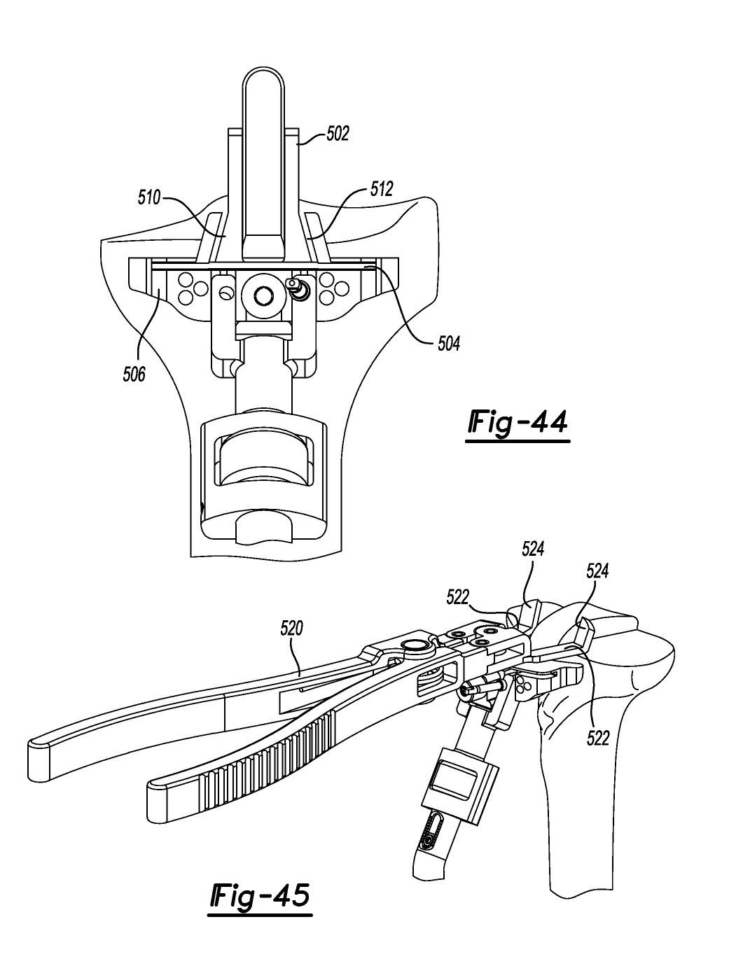

Turning now to FIG. 44, a cut guide 502 can be adjusted along a slot 504 of a tibial resection block 506. In general, the vertical cut guide 502 and the tibial resection block 506 can be formed similar to the vertical cut guide 72 and tibial resection cut block 70 described above and shown in FIG. 12. However, the cut guide 502 can define a medial cut slot 510 and a lateral cut slot 512 that are formed at an angle corresponding to the resulting medial chamfer wall 372 and lateral chamfer wall 382 (FIG. 38).

FIG. 45 illustrates a Rongeur tool 520 having a pair of cutting arms 522 that each have a corresponding pair of distal cutting tips 524. The distal cutting tips 524 can be formed at a generally obtuse angle relative to the arms 522 such that actuation of the Rongeur tool 520 can cause the cutting tips 524 to move toward each other and create the anterior chamfer wall 486 illustrated in FIG. 41. It will be appreciated that the tools shown in FIGS. 44 and 45 are merely exemplary and other tools may be used.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A tibial tray configured for use in a bi-cruciate retaining knee procedure, the tibial tray comprising:
a medial portion having a superior medial bearing opposing surface, an inferior medial bone opposing surface and a medial connecting surface disposed between the superior medial bearing opposing surface and the inferior medial bone opposing surface;
a lateral portion having a superior lateral bearing opposing surface, an inferior lateral bone opposing surface and a lateral connecting surface disposed between the superior lateral bearing opposing surface and the inferior lateral bone opposing surface;
an anterior connecting portion that connects the medial and lateral portions and cooperates with the medial and lateral portions to define a slot between the medial and lateral portions for accommodating at least one of a natural and replacement anterior cruciate ligament and a posterior cruciate ligament;
wherein the medial and lateral connecting surfaces are formed on converging planes that extend from respective inferior bone opposing surfaces to superior bearing opposing surfaces.

2. The tibial tray of claim 1 wherein the medial connecting surface is chamfered relative to the superior medial bearing opposing surface.

3. The tibial tray of claim 2 wherein the medial connecting surface defines an angle of between 30 degrees and 60 degrees relative to the superior medial bearing opposing surface.

4. The tibial tray of claim 3 wherein the medial connecting surface defines an angle of about 45 degrees relative to the superior medial bearing opposing surface.

5. The tibial tray of claim 2 wherein the lateral connecting surface is chamfered relative to the superior lateral bearing opposing surface.

6. The tibial tray of claim 5 wherein the lateral connecting surface defines an angle of between 30 degrees and 85 degrees relative to the superior lateral bearing opposing surface.

7. The tibial tray of claim 6 wherein the lateral connecting surface defines an angle of about 45 degrees relative to the superior lateral bearing opposing surface.

8. The tibial tray of claim 1 wherein the anterior connecting portion has an inferior anterior bone opposing surface and further includes an anterior connecting surface that extends at an obtuse angle relative to the inferior anterior bone opposing surface.

9. The tibial tray of claim 8 wherein the anterior connecting surface extends at an angle between 120 degrees and 150 degrees relative to the inferior anterior bone opposing surface.

10. The tibial tray of claim 1 wherein the medial and lateral connecting surfaces are configured to overhang respective medial and lateral chamfer walls formed on an anterior cruciate island of a tibia in an implanted position.

11. A tibial tray configured for use in a bi-cruciate retaining knee procedure, the tibial tray comprising:
a medial portion having a superior medial bearing opposing surface, an inferior medial bone opposing surface and a medial connecting surface disposed between the superior medial bearing opposing surface and the inferior medial bone opposing surface;
a lateral portion having a superior lateral bearing opposing surface, an inferior lateral bone opposing surface and a lateral connecting surface disposed between the superior lateral bearing opposing surface and the inferior lateral bone opposing surface;
an anterior connecting portion that connects the medial and lateral portions and cooperates with the medial and lateral portions to define a slot between the medial and lateral portions for accommodating at least one of a natural and replacement anterior cruciate ligament and a posterior cruciate ligament;
wherein the anterior connecting portion has an inferior anterior bone opposing surface and further includes an anterior connecting surface that extends at an obtuse angle relative to the inferior anterior bone opposing surface.

12. The tibial tray of claim 11 wherein the anterior connecting surface extends at an angle between 120 degrees and 150 degrees relative to the inferior anterior bone opposing surface.

13. The tibial tray of claim 11 wherein the medial and lateral connecting surfaces are formed on converging planes that extend from respective inferior bone opposing surfaces to superior bearing opposing surfaces.

14. A method for preparing a proximal tibia for receipt of a bi-cruciate implant, the method comprising:
determining a resection level of the proximal tibia, the resection level corresponding to a medial and lateral tibial plateau;
preparing a medial cut into the proximal tibia, the medial cut having an obtuse angle relative to the medial tibial plateau, the medial cut creating a medial chamfer wall on the proximal tibia; and
preparing a lateral cut into the proximal tibia, the lateral cut having an obtuse angle relative to the lateral tibial plateau, the lateral cut creating a lateral chamfer wall on the proximal tibia.

15. The method of claim 14, further comprising:
positioning a tibial tray onto the prepared proximal tibia wherein (i) a medial connecting surface of the tibial tray overhangs the medial chamfer wall of the proximal tibia and (ii) a lateral connecting surface of the tibial tray overhangs the lateral chamfer wall of the proximal tibia.

16. The method of claim 15, further comprising disposing bone cement (i) between the medial chamfer wall and the medial connecting surface and (ii) between the lateral chamfer wall and the lateral connecting surface.

17. The method of claim 14, further comprising:
fixing a tibial cut block relative to the proximal tibia based on the determination;
slidably translating a vertical cut guide along a slot defined in the tibial cut block until a desired medial-lateral position relative to the proximal tibia has been attained; and
fixing the vertical cut guide relative to the tibial cut block based on attaining the desired medial-lateral position.

18. The method of claim 16 wherein slidably translating comprises locating a tongue extending from the vertical cut block into the slot of the tibial cut block.

19. The method of claim 18 wherein fixing the vertical cut guide comprises moving a locking arm extending from the vertical cut guide from an unlocked position to a locked position, wherein in the locked position, a finger extending from the arm engages the tibial cut block.

20. The method of claim 19 wherein moving the locking arm comprises rotating the locking arm relative to the vertical cut guide from the unlocked position to the locked position.

21. The method of claim 14, further comprising preparing an anterior cut into the proximal tibia, the anterior cut creating an anterior chamfer wall on the proximal tibia.

22. The method of claim 21 wherein preparing the anterior cut comprises:
positioning a Rongeur tool having cutting tips that define obtuse angles relative to corresponding cutting arms relative to an anterior portion of the tibia; and
actuating the cutting arms wherein the cutting tips cut the bone and prepare the anterior cut.
